(19) United States
(12) Patent Application Publication
(10) Pub. No.: US 2004/0209278 A1
(43) Pub. Date: Oct. 21, 2004

(54) Detection of modular polyketide synthase gene clusters

(75) Inventors: Andreas Schirmer, Hayward, CA (US); Peter Revill, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kosan Biosciences, Inc., Hayward, CA (US)

(21) Appl. No.: 10/676,689

(22) Filed: Sep. 30, 2003

Related U.S. Application Data
(60) Provisional application No. 60/415,305, filed on Sep. 30, 2002.
(60) Provisional application No. 60/415,326, filed on Sep. 30, 2002.

(51) Int. Cl. C12Q 1/68; C12P 19/34

(52) U.S. Cl. 435/6; 435/91.2

(57) ABSTRACT

A PCR based method is provided by which DNA and cDNA of a polyketides-producing host can be rapidly queried to identify sequences of a target gene.

### CROSS REFERENCE TO RELATED APPLICATIONS

This application claims benefit of U.S. provisional patent applications 60/415,305 and 60/415,326, both filed Sep. 30, 2002, the entire disclosures of which are incorporated herein by reference.

### FIELD OF THE INVENTION

The present invention relates to a methods of detecting polynucleotide sequences and genes in a producer cell encoding polyketide synthase (“PKS”) enzymes. The invention relates to the fields of molecular biology, chemistry, recombinant DNA technology, medicine, animal health, and agriculture.

### BACKGROUND OF THE INVENTION

Polyketides represent a large family of diverse compounds synthesized from two-carbon units through a series of condensations and subsequent modifications. Polyketides occur in many types of organisms including fungi and mycelial bacteria, in particular the actinomycetes. An appreciation for the wide variety of polyketide structures and for their biological activities, may be gained upon review of the extensive art, for example, published International Patent Specification WO 95/08548; U.S. Pat. Nos. 5,672,491 and 6,303,342; and the journal articles H. Fu et al., Biochemistry, 33, pp. 9321-9326, (1994); R. McDaniel et al., Science, 262, pp. 1546-1550, (1993); and J. Rohr, Angew. Chem. Int. Ed. Engl. 34(8), pp. 881-888, (1995).

Polyketides are synthesized in nature by polyketide synthases (“PKS”). Two major types of PKS are known and differ in their mode of synthesis. These are commonly referred to as Type I or “modular” and Type II “iterative.” The Type I or modular PKS comprise a set of separate catalytic activities; each activity is termed a “domain”, and a set thereof is termed a “module”. One module exists for each cycle of carbon chain elongation and modification. WO95/08548 depicts a typical Type I PKS, in this case 6-deoxyerythronolide B synthase which is involved in the production of erythromycin.

Cloning of a novel PKS gene cluster faces two major problems: (i) the genomes of PKS producing organisms usually contain multiple PKS clusters and (ii) different PKS cluster are very similar in structure and sequence (70-90% sequence identity at the DNA level are not uncommon). Therefore probes for cloning a particular PKS gene cluster are often not very specific for the target PKS cluster but rather tend to be generic and can hybridize to any PKS cluster in the genome. As a consequence, extensive sequencing of PKS clones or whole genomes is necessary to identify and isolate a target PKS cluster. These procedures can be very costly and time consuming.

Usually, a target PKS cluster from a given microorganism is cloned by hybridization of genomic libraries with one or a few PKS amplimers isolated by degenerate PCR from genomic DNA of this organism. The major disadvantage of this approach is that the isolated PKS amplimer(s) might not be part of the target PKS gene cluster. Therefore, these probes might hybridize more strongly to non-target PKS clusters and might even fail to hybridize with the target PKS cluster.

There is therefore a need for methods of detecting those nucleic acids in host cells that produce polyketides and result in the targeted cloning of polynucleotides encoding synthases and modifying enzymes to produce polyketide compounds at a commercially useful scale and to make polyketides analogs. These and other needs are met by the materials and methods provided by the present invention.

### SUMMARY OF THE INVENTION

In one aspect, the invention provides a method for obtaining a probe that hybridizes to a gene in a PKS gene cluster by (a) identifying amplimers produced at higher frequency from amplification of cDNA from RNA of a producer cell and degenerate PCR primers that hybridize to consensus regions of gene sequences encoding a PKS domain, compared to amplification of genomic DNA of the producer cell using the same primers; and, (b) using the sequences of the amplimers selected in (a) for designing one or more probes for cloning genes in a PKS gene cluster. In some embodiments, the PKS domain is KR, AT, ACP, KR, DH, ER, or TE (or more than one domain). In some embodiments, the cDNA is prepared from RNA collected at least two different times and/or from RNA collected from cells cultured under at least two different production conditions. In an embodiment, the cDNA is prepared from RNA from cells collected prior to the time of maximum polyketide production. In a related aspect, a probe designed using this method is used to screen a genomic DNA library of the producer cell for clones comprising sequence of a gene in a PKS gene cluster. In a related aspect, the invention provides a method for detecting a nucleic acid encoding a PKS gene by hybridizing a probe obtained by this method to said nucleic acid and detecting the hybridization complex.

In another aspect, the invention provides a method for obtaining a probe that hybridizes to a gene encoding a first PKS gene by (a) determining the sequences of a plurality of amplimers prepared using degenerate PCR primers that hybridize to consensus regions of gene sequences encoding a PKS domain; (b) determining phylogenetic similarity for the amplimers in (a) and plurality of sequences encoding a domains of a gene or genes encoding one or more PKS related to said first PKS; (c) selecting the amplimer sequences from (a) that are most closely related to one or more domain-encoding sequences in (b); and, (d) using the sequences selected in (c) for designing probes that hybridize to said first PKS gene. In embodiments, the domain is KR, AT, ACP, KR, DH, ER, or TE (or more than one domain). In an embodiment, determining phylogenetic similarity is done using a computer running ClustalW software. In an embodiment, the sequence of the first PKS gene is not known. In a related aspect, the invention provides a method for detecting a nucleic acid encoding a PKS gene comprising hybridizing a probe obtained by this method to the nucleic acid and detecting the hybridization complex.

### BRIEF DESCRIPTION OF THE DRAWINGS

FIG. 1 shows a chart of a hypothetical experiment in which amplimers corresponding to 10 KS domains were obtained by PCR of genomic DNA or cDNA from a producer cell. Each occurrence of an amplimer in the cDNA pool is indicated by an “x” and each occurrence of an amplimer in the genomic pool is indicated by an “o.”

FIG. 2 shows the frequencies of FK520 KS domain amplimers from total RNA (different time points) versus genomic DNA (FIG. 1B). The production curves of the Streptomyces hygroscopicus cultures used for RNA isolation are also shown (FIG. 1A). The times when RNA was prepared are marked with arrows.

FIG. 3 shows a PKS similarity tree of Streptomyces hygroscopicus ATCC14891. FK520 KS sequences are bold.

FIG. 4 shows a PKS similarity tree of Streptomyces bikiniensis. All unique KS DNA sequences were aligned with Tylosin KSs (Tylosin KSs: bold and italicized, putative Chalcomycin KSs: bold)

FIG. 5 shows a PKS similarity tree of Micromonospora chalcea. All unique KS DNA sequences were aligned with Tylosin KSs (Tylosin KSs: bold and italicized, putative Juvenimycin KSs: bold and italicized).

### DETAILED DESCRIPTION OF THE INVENTION

I. Introduction

The invention provides methods for targeted cloning of a specific PKS cluster from a organism with multiple PKS gene clusters. Historically, novel PKS clusters were cloned by hybridization of cosmid libraries with heterologous PKS probes, with the DEBS PKS genes being the most widely used. In general this approach suffers from the possibility that a heterologous probe can hybridize more efficiently to a non-target PKS sequence than to the target PKS gene. (As used herein, the “target” gene(s) are those uncloned genes of interest for which specifically hybridizing probes and primers are sought).

After a considerable number of PKS gene sequences became available for deduction of consensus sequences, the use of degenerate primers to amplify conserved KS, AT or KR sequences from the producer organism using polymerase chain reaction (PCR) techniques was frequently adopted. Using these methods, a few PCR products (“amplimers”) are cloned, sequenced and used as homologous probes for hybridization of a library. However, a major disadvantage of these approaches is that the target PKS cluster might not be contained within the probe pool.

The present invention provides methods for the rapid detection, identification, and isolation for targeted cloning of DNA molecules that comprise one or more coding sequences for one or more domains or modules of polyketide synthases or PKS related genes. Examples of such encoded domains include ketosynthase (KS), acyltransferase (AT), acyl carrier protein (ACP), ketoreductase (KR), dehydratase (DH), and enoylreductase activity (ER) domains. PKS related genes are biosynthetic genes that produce PKS starter units or extender units (e.g., AHBA synthases), polyketide modifying enzymes (e.g., oxygenases, glycosyl- and methyltransferases, acyltransferases, halogenases, cyclases, aminotransferases, and hydroxylases), and non-ribosomal peptide synthases (NRPSs).

In one aspect of the invention, PCR amplimers are prepared using degenerate primers using cDNA prepared from RNA of the target organism and changes in the frequency of particular amplimers is used to identify PKS genes of interest.

In a different aspect of the invention, PCR amplimers are prepared using degenerate primers and cluster analysis is carried out using known PKS sequences for comparison.

In a different aspect of the invention, PCR amplimers are prepared using degenerate primers and cluster analysis is carried out using known amplimers from a related strain for comparison.

Each of these aspects is described in greater detail below.

II. Analysis of Amplicons from cDNA

The invention provides a method for obtaining a probe that hybridizes to a gene in a PKS gene cluster by (a) identifying amplimers produced at higher frequency from amplification of cDNA from RNA of a producer cell and degenerate PCR primers that hybridize to consensus regions of gene sequences encoding a PKS domain, compared to amplification of genomic DNA of the producer cell using the same primers; (b) using the sequences of the amplimers selected in (a) for designing one or more probes for cloning genes in a PKS gene cluster. As used herein, a “producer” cell is a cell that makes a polyketide of interest. It is generally the object of the investigator to clone the gene cluster encoding the PKS that produces the polyketide of interest.

In one embodiment, the method of the invention involves (a) determining the sequence of amplimers prepared using (i) degenerate PCR primers that hybridize to consensus regions of PKS gene domains, and (ii) cDNA prepared from RNA of the producer cell; and, (b) for each amplimer in (a), comparing its frequency of appearance in (a) with the frequency of appearance in a set of amplimers obtained from genomic DNA of the producer using the same primers. Sequences from amplimers that appear with higher frequency in the cDNA pool than in the genomic pool are used to design specific probes or primers for cloning genes in a PKS gene cluster.

Methods for preparation of cDNA from producer cells are well known and vary to some degree with the nature of the producer cell. Generally, RNA is prepared under conditions that minimize degradation. Such techniques are explained fully in the literature, such as Current Protocols in Molecular Biology (Ausubel et al., eds., 1987, including supplements through 2001); Molecular Cloning: A Laboratory Manual, third edition (Sambrook and Russel, 2001). Generally total RNA is used, but it is possible to use an RNA fraction (i.e., an RNA-minus fraction). The purified RNA is reverse-transcribed to produce either a single- or double-stranded cDNA. The cDNA can be used as a template for PCR using degenerate primers corresponding to conserved regions of genes encoding PKS domains (or conserved regions of other target genes). The domain type corresponding to the conserved regions can be called the “expected amplification domain.” Thus, for example, KS domains are the expected amplification domains for degenerate primers corresponding to conserved regions of KS domains, AT domains are the expected amplification domains for degenerate primers corresponding to conserved regions of AT domains, etc.

PCR amplification reactions are conducted using cDNA prepared from RNA (e.g., reverse-transcription PCR), and on genomic DNA from the producer cell. Methods for PCR amplification are well known (see, e.g., Ausubel, supra). The design of degenerate primers corresponding to conserved regions of genes encoding PKS or other domains is known in the art and is described in the examples, below.

According to the invention, PCR is also carried out using genomic DNA from the producer organism as template, and using the same sets of degenerate PCR primers as used for the cDNA. It is not necessary to do the genomic and cDNA amplifications are the same time. For example, the genomic amplification can be done first and the results recorded for later comparison with cDNA results.

The products of a polymerase chain reaction, or amplimers, obtained from the genomic and cDNA PCR step are sequenced. Any DNA sequencing method can be used. To identify amplimers corresponding to a target PKS, usually at least 25 different amplimers are sequenced, often at least 50 amplimers are sequenced, and it is not unusual to sequence at least 100 different amplimers. In general, using larger numbers of amplimers will give the most reliable results.

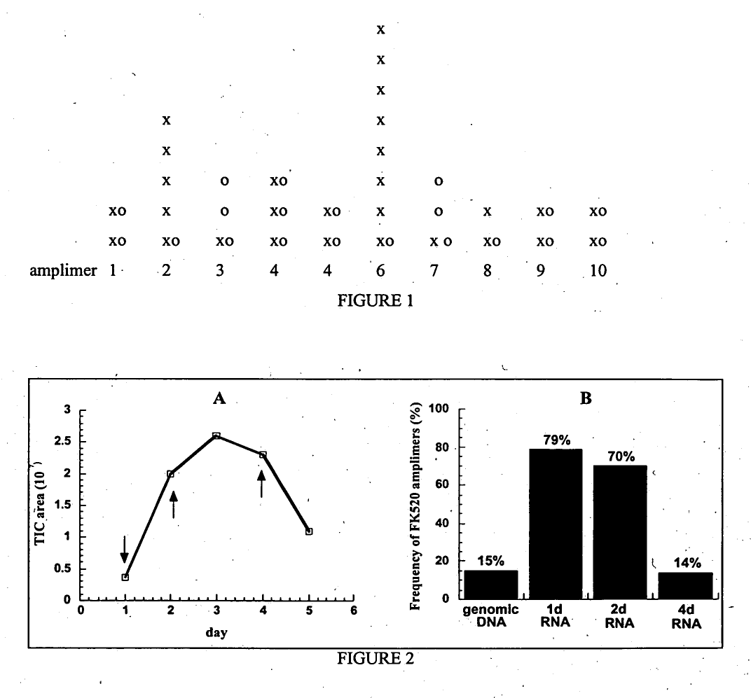

The frequency of appearance of amplimer sequences from the RNA template vs. the genomic DNA template is compared. This can easily be done by preparing a chart as shown in FIG. 1. Optionally, spurious amplimers (i.e., sequences not from the expected amplification domains) can be removed prior to the comparison. Those sequences for which there is the greatest increase in frequency when comparing the cDNA vs. genomic amplifications are more likely than other amplimers to correspond to the gene (e.g., PKS gene) of interest. Probes or primers can be designed based on the sequences of the amplimers, or the amplimers themselves can be labeled and used as probes (by “designed” is meant that probe and primer sequences hybridize to the amplimer sequences or their complements). Alternatively, the amplimer sequences can be used to search sequence databases. Uses of the probes obtained by the methods of the invention are described below in Section IV.

In another embodiment of the invention, cDNA is made from RNA obtained from cells culture for different lengths of time (e.g., by sampling from a culture at different times). See Example 3, below. In one embodiment of the invention, cDNA is made from RNA obtained prior to the time of maximum production of the polyketide of interest.

In another embodiment of the invention, cDNA is made from RNA from cells growing under different production conditions (e.g., conditions of high production of a polyketide or low production);

Example 3, below, shows the application of this method in a model system, Streptomyces hygroscopicus ATCC14891, the producer of FK520.

III. Cluster Analysis of Producer Amplicons With Known Sequences and With Each Other

In another aspect, the invention provides a method for obtaining a probe that hybridizes to gene encoding a PKS gene of sequence (i.e., a PKS gene that produces a polyketide of interest). The method involves determining the sequences of a plurality of amplimers prepared using degenerate PCR primers that hybridize to consensus regions of gene sequences encoding a PKS domain; generating a phylogenetic similarity tree for the amplimers and for a plurality of sequences encoding domains of PKS genes encoding synthases related to said first PKS; selecting the amplimer sequences that are most closely related to one or more domain-encoding sequences from the related PKS genes. Either genomic DNA or cDNA can be used in this method. The selected sequences can used to design probes and primers, or the amplimers themselves can be labeled and used as probes. Alternatively, the amplimer sequences can be used to search sequence databases.

As noted above, the design of degenerate PCR primers is known in the art, as is illustrated in the examples, below. The number of amplimers sequenced can vary, but to identify amplimers corresponding to a target PKS, usually at least 25 different amplimers are sequenced, often at least 50 amplimers are sequenced, and it is not unusual to sequence at least 100 different amplimers. As noted above, in general, using larger numbers of amplimers will give the most reliable results.

Unique amplimer sequences with sequences related to the expected amplification domains (e.g., KS domains) are identified and phlylogenetic similarity is determined for the unique amplimer sequences and corresponding sequences of related PKS genes. “Unique” in this context, means that only a single sequence is used for each domain even though multiple amplimer sequences can be generated (e.g., cloned) from each domain. (Multiple amplimer sequences from the same domain can have different sequences if different degenerate primer pairs are used or due to small differences introduced during amplification and cloning.) The term “related PKS genes” means PKS genes responsible for the biosynthesis of a polyketide(s) whose chemicals structure(s) resemble the target polyketide. The similarity in structure typically refers to a common carbon backbone, a common starter unit and/or a common modification. Type I polyketides have been grouped into several classes according to these similarities (for a recent review see: Rawlings, 2001, Type I polyketide biosynthesis in bacteria, Nat. Prod. Rep., 18:231-81). This similarity in polyketide structure often corresponds to similarities in sequence and/or gene structure in the corresponding polyketide synthase genes. These similarities are thought to reflect evolutionary (or phylogenetic) relationships; i.e., for a particular class of polyketides, a common ancestral PKS gene might have diverged to synthesize different polyketides within this class, leading to the observed sequence and gene structure relationships of the PKS genes, and the observed structural similarities of the polyketides.

Many classes of type I polyketides have members for which the sequence of a biosynthetic gene cluster is known. These include: 14-membered macrolides (e.g. Erythromycin, Pikromycin, Oleandomycin, Megalomicin), 16-membered macrolides (e.g. Tylosin, Niddamycin, Spiramycin, Mycinamicin), Ansamycins (e.g. Rifamycin, Geldanamycin, Ansamitocin), Polyenes (e.g. Nystatin, Amphotericin B, Pimaricin), Polyethers (e.g. Monensin, Nanchangmycin), Rapamycin and related compounds (Rapamycin, FK520, FK506), Avermectins and related compounds (e.g. Avermectin, Oligomycin). There are other polyketides for which the sequence of a biosynthetic gene cluster is available but that are not yet commonly categorized as part of a class because few or no polyketides similar in structure have as yet been identified (e.g. the Spinosyns, the Epothilones, Soraphen, Spirangiene, Stigmatellin, Myxalamid, Myxothiazol). There exist other commonly used classes for which as yet no sequence of a biosynthetic gene cluster is available (e.g., the Hygrolidin/Bafilomycin-related group).

PKS genes that are known to be responsible for the biosynthesis of polyketides whose chemical structure resembles the target polyketide, e.g. Chalcomycin resembles Tylosin (both are 16-membered macrolides) and Geldanamycin resembles Ansamytosin and Rifamycin (all three are ansamycins).

Phylogenetic similarity can be determined by creating phylogenetic similarity “trees.” The invention makes use of phylogenetic similarity trees for identification of domain sequences of interest. Phlylogenetic similarity trees (in the present context) are graphic or mathematical representations of similarities between multiple DNA sequences showing the degree of sequence identities of multiple related sequences. Such trees can be generated using a variety of methods. Generally, the widely available computer program CLUSTALW is used for alignments. (Thompson et al., 1994, CLUSTALW: improving the sensitivity of progressive multiple sequence alignment through sequence weighting, position specific gap penalties and weight matrix choice. Nucleic Acids Research, 22:4673-4680; Higgins et al., 1996, Using CLUSTAL for multiple sequence alignments, Methods Enzymol 266:383-402) with output based on the PHYLIP program of Felenstein and the (Felsenstein, J., 1985, Confidence limits on phylogenies: an approach using the bootstrap. Evolution, 39:783-791; Felsenstein, J., 1988, Phylogenies from molecular sequences: Inference and reliability Annu. Rev. Genet., 22:521-565; Felsenstein, J., 1990, PHYLIP manual, version 3.3 University of Washington, Seattle; Felsenstein, J., 1993, PHYLIP manual, version 3.5 University of Washington, Seattle) can be used for similarity analysis. In one embodiment ClustalW is used, with the default parameters, from the MacVector version 6.5.3 sequence analysis package (Accelrys, San Diego)) Other methods for generation of phylogenetic trees include TreeAlign (Hein, 1990, Methods Mol Biol. 25:349-64) MALIGN (Wheeler and Gladstein, 1994, J. Hered 85:417-18) and SAM 1.1a (Hughey and Krogh, 1996, Comp. Appl Biosci 12:95-107). It will be appreciated that “generating a phylogenetic similarity tree” does not require output of a graphical tree representation of the relationship between amplimer sequences (although such graphical representations are useful) if other types of representations are preferred.

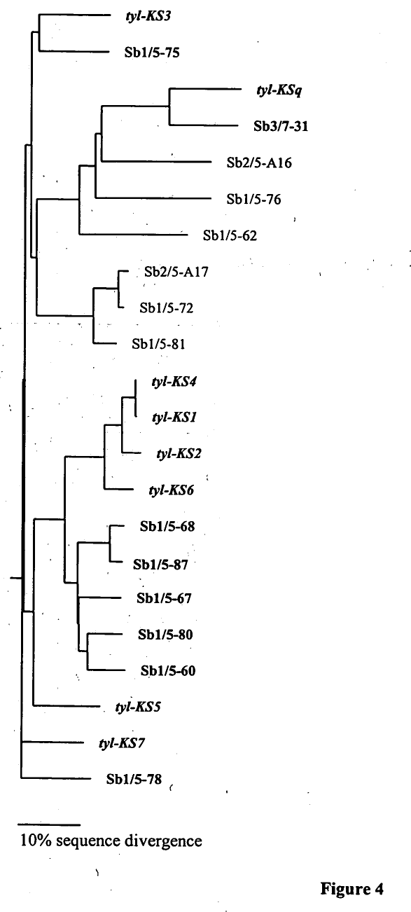

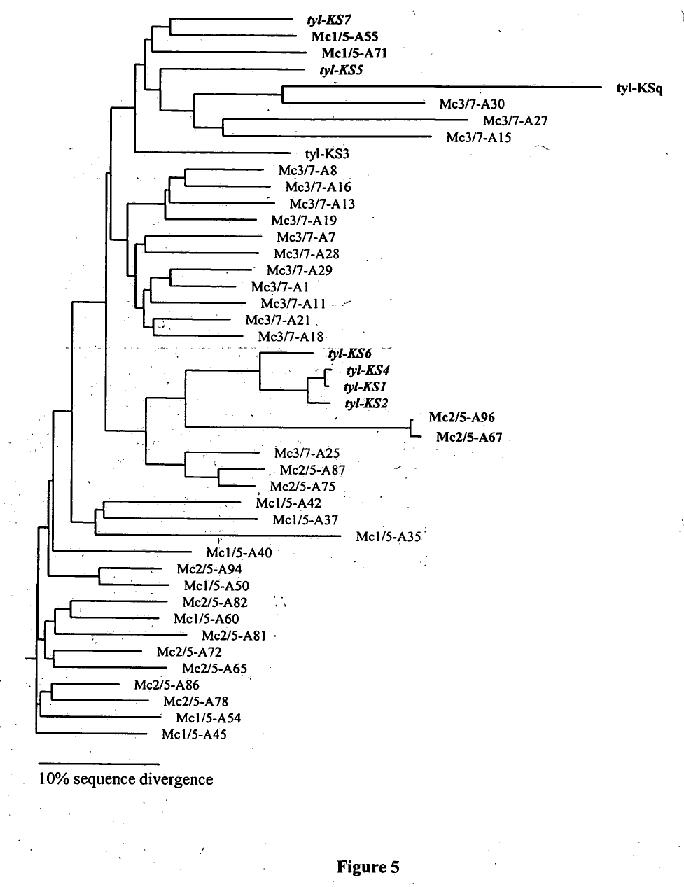

In one aspect, a target PKS cluster can be identified by comparing the phylogenetic PKS similarity tree of a producer strain with the corresponding KS sequences of related PKS genes from a different strain: For example, if within a complex PKS tree of a given strain, a subset of KS sequences clearly clusters together with KS sequences of a related PKS cluster from a different strain, then these KS sequences are likely to be part of target PKS gene cluster in this strain (see FIGS. 4). FIG. 4 shows a PKS similarity tree of Streptomyces bikiniensis (chalcomycin producer) comparing all unique amplimer (KS domain) sequences aligned and tylosin ketosynthase KS domain sequences, and showing that some amplimer sequences are clustered together with tylosin sequences. For example, amplimer Sb3/7-31 and Tylosin KSq appear to have a common ancestral sequence with about 11% sequence divergence. Furthermore this amplimer shows less than 20% sequence divergence to Tylosin KSq, but more than 35% divergence to its most closely related KS amplimer in the S. bikiniensis genome. FIG. 5 shows a phylogenetic tree of Micromonospora chalcea (juvenimycin producer), comparing all unique amplimer (KS domain) sequences and tylosin ketosynthase KS domain sequences. This procedure is useful when DNA sequences of related PKS are available in sequence databases.)

Once candidates have been identified, they then can then be compared to all known KSs in the database (e.g., using BlastX and GenBank). If they have the chosen related PKS cluster as the best match, this confirms that they were correctly chosen (i.e. they are not only very similar to the chosen related sequence in the PKS tree, but also less similar to all other known KS sequences).

In another aspect of the invention, all unique amplimer sequences of a given strain are compared with each other in a phylogenetic PKS similarity tree. FIG. 2 shows the grouping of FK520 PKS amplimers from the producing host cell Streptomyces hygroscopicus ATCC14891. Note that the FK520 KS amplimers form a distinctive cluster within the PKS tree of this strain, indicating that the phylogenetic clustering of KS sequences can correspond to distinctive PKS gene clusters in the genomes of the producer strains. Thus, this procedure (i) gives a measure of the number and nature of the PKS gene clusters of this strain, (ii) identifies unique KS sequences and (iii) assigns KS amplimers to phylogenetic clusters correlated with individual separate PKS gene clusters within the genome of the strain.

IV. Cluster Analysis of Amplicons of Two Related Species

In a different embodiment, amplimers are produced from different species of organism that each produce a polyketide that is the same or structurally similar to a polyketide produced by the other. The amplimers are sequenced and a PKS similarity tree is produced using unique sequences. Amplimer sequences from the two species that cluster together are likely candidates for target genes. This procedure does not require prior knowledge of related PKS gene clusters. In this case, KS sequences that are similar or identical in both strains are likely candidates for target PKS genes.

V. Further Steps

Once target PKS gene sequences are identified they can be applied (i) to isolate target PKS clones and (ii) to design specific primers to verify target PKS clones before sequencing. It should be emphasized that increasing the number of KS amplimers analyzed for a given strain will increase the likelihood for success, because the method relies on obtaining a representative set of PKS gene fragments of a given strain.

Probes and primers based on amplimer sequences identified using the methods of the invention of can be used for amplification of producer cell sequences. Alternatively, they can be labeled and used as probes that can be hybridized to a complementary and the hybridization complex detected. Methods for labeling and hybridization are well known (see, e.g., Ausubel). Many other uses of amplimer sequences (e.g., use for targeted knock-out by homologous recombination, use to design immunogens) will be apparent to those of skill guided by this disclosure.

In one aspect, the invention provides a method to isolate a modular polyketide synthase (PKS), modifying or precursor gene in a producer cell DNA by designing degenerate PCR primers that hybridize to consensus regions of known PKS gene domains, constructing a host cell DNA library, performing a PCR reaction using the degenerate primers on the producer cell DNA library, isolating amplimer products from said PCR reaction, sequencing the amplimers, performing a similarity analysis of the amplimer sequences with known PKS gene sequences, identifying the modular PKS genes of interest, designing specific probes to the modular PKS genes of interest using the amplimer sequences, probing the producer DNA library with the amplimer specific probes, identifying DNA library clones containing the modular PKS genes, and cloning the gene sequences.

The methods of the present invention have been applied to (1) identify the geldanamycin PKS, gene cluster; (2) identify AHBA precursor synthesis cluster; (3) to test PCR primers and conditions with multiple PKS gene cluster encoding microorganisms in Streptomyces hygroscopicus ATCC14893 producing FK520 and Sorangium cellulosum Soce90 producing epothilone; and (4) to identify and clone novel 16-membered PKS genes in Micromonospora chalcea ATCC21561 producing juvenimicin) and Streptomyces bikiniensis NRRL2737 producing chalcomycin.

Thus, the present invention provides methods to rapidly query and identify the presence of type I modular PKS genes, then the number of these genes and their individual characteristics can be established by DNA sequences and bioinformatics analysis of short PKS amplimers.

### EXAMPLES

### Example 1

### Methods

This Example described experimental methods used in Examples 2-4.

A. Growth Conditions for RNA Isolation

For RNA isolation, Streptomyces hygroscopicus ATCC14891 and Sorangium cellulosum Soce90 were grown in their respective polyketide production media. S. hygroscopicus: tryptone soya broth 3%, glucose 1%, pH adjusted to 6.0 with 2-[morpholino]ethansulfonic acid. S. cellulosum: potato starch 0.8%, yeast extract 0.2%, soybean flour 0.2%, Fe(III)EDTA 0.0008%, MgSO₄×7 H₂O 0.1%, CaCl₂×2 H₂O 0.1%, HEPES 1.15%, glucose 0.2%, pH adjusted to 7.4 with KOH.

B. RNA Isolation, RT-PCR, and Degenerate PCR

Total RNA from S. hygroscopicus and S. cellulosum was prepared using standard methods. A two-step RT-PCR was developed using the Thermoscript™ RT-PCR system (Invitrogen): cDNA synthesis was typically done with 2-5 μg of total RNA and 50 ng/μl of random hexameric primers for 10 min at 25° C. followed by 50 min at 50° C. in a 20 μl volume. 2-4 μl of cDNA was then used as template for PCR with 200 pmol of degenerate primers and 2U of Taq DNA polymerase (Boehringer) in a 50 μl volume.

RT-PCR was carried out using primers degKS2F+5R and degKS3F+7R. PCR products were cloned and between 30 and 40 for each primer pair were sequenced.

### Example 2

### The Use of RNA and RT-PCR to Obtain Specific PKS Gene Probes

As shown in FIG. 2B, the number of FK520 amplimers from RNA of S. hygroscopicus isolated at two days is greater than amplimers from genomic DNA.

The amplimer sequences were compared to the NCBI database using BLAST to identify Type I KS sequences in general and FK520 KS sequences in particular. The frequency of FK520 KS amplimers relative to the total number of KS amplimers with the two different degenerate primer pairs was determined and compared for the use of genomic DNA or total RNA as template.

Remarkably, the frequency of FK520 amplimers using RNA rose up from 7% (DNA) to 64% for degKS2F+5R and from 15% (DNA) to 80% for degKS3F+7R (see Table 1). The FK520 gene cluster contains 10 different ketosynthases (Wu et al., 2000, The FK520 gene cluster of Streptomyces hygroscopicus var. ascomyceticus (ATCC 14891) contains genes for biosynthesis of unusual polyketide extender units, Gene 251: 81-90). All but FK520 KS9 and KS10 were amplified from RNA with KS3 (7×), KS7 (4×) and KS1, 2, and 4 (each 3×) most frequently found. A certain bias of the primers for individual KS sequences is not unexpected, but there is clearly no strong bias for FK520 KSs in general (otherwise the numbers would be similarly high with both DNA and RNA) and the results can only be explained with an overabundance of FK520 relative to other PKS mRNAs under the chosen conditions.

TABLE 1
Comparison of number and frequency of PCR and RT-PCR
amplimers generated from S. hygroscopicus DNA and RNA
using degenerate KS primers

| | Primers degKS2F + 5R | | | | Primers degKS3F + 7R | | | |
|---|---|---|---|---|---|---|---|---|
| amplimers | DNA | Freq. | RNA | Freq. | DNA | Freq. | RNA | Freq. |
| # total | 17 | | 40 | | 31 | | 36 | |
| # PKS | 15 | 87%¹ | 11 | 28%¹ | 27 | 87%¹ | 30 | 83%¹ |
| # FK520 | 1 | 7%² | 7 | 64%² | 4 | 15%² | 24 | 80%² |

¹compared to total number of sequences
²compared to number of PKS sequences

### Example 3

### Frequency of FK520 Amplimers from RNA of S. hygroscopicus in a Time Course Experiment

To determine if the relative abundance of FK520 transcripts corresponds to the titer of FK520 in the culture, we monitored the production of FK520 over time and prepared RNA at days 1, 2 and 4. RT-PCR was performed with primers degKS3F+7R and 20-30 RT-PCR products for each time point were analyzed. FIG. 2A shows the production curve of FK520 and FIG. 2B shows the frequency of FK520 amplimers during the time course experiment. The frequency of FK520 amplimers with degKS3F+7R from genomic DNA was previously determined to be 15% (see above). The frequency from RNA was found to be significantly increased to 79% and 70% from RNA at day one and two, respectively, confirming the results of the earlier experiment. At day four however, the frequency was down again to 14%, which is comparable to genomic DNA. Apparently, the maximum of FK520 transcripts slightly precedes the maximum of FK520 production. These date indicated that RNA isolated at such an early time point of a target polyketide production curve is a good source for production of specific probes.

### Example 4

### Frequency of Epothilone Amplimers from RNA of S. cellulosum in a Time Course Experiment

In an analysis of a second organism, the epothilone producer Sorangium cellulosum Soce90, the frequency of epothilone amplimers from genomic DNA using primers degKS3F+7R.mx was determined to be 30% (10 out of 32). We monitored the production of epothilone over time (maximum at day 6) and prepared RNA at day 2, 4 and 6 and analyzed 20-30 RT-PCR products. In this study, the frequency of epothilone PKS amplimers did not differ significantly between genomic DNA and RNA from any of the different time points (between 20% and 30%). In contrast to FK520 transcripts from S. hygroscopicus, there was apparently no significant overabundance of epothilone transcripts relative to other PKS transcripts at early time points in S. cellulosum.

### Example 5

### Design and Testing of Degenerate PKS Primer with Streptomyces hygroscopicus ATCC14893 and Sorangium cellulosum SOCE90

Six degenerate PCR primers were designed based on conserved regions of ketosynthase (KS) domains of type I PKS genes and codon bias of actinomycetes (see Table 2). These primers were tested with genomic DNA of Streptomyces hygroscopicus ATCC14893 in the following combinations: degKS1F+5R, degKS1F+KS6R, degKS2F+5R and degKS3F+7R. Four degenerate PCR primers were designed based on conserved regions of ketosynthase (KS) domains of type I PKS genes and codon bias of myxobacteria (see Table 3). These primers were tested with genomic DNA of Sorangium cellulosum Soce90 in the following combinations: degKS1F.mx+5R.mx and degKS3F.mx+7R.mx. The PCR conditions for the amplification of KS domains were as follows: A total reaction volume of 50 μl contained 100 ng of genomic DNA, 200 pmol of each primer, 0.2 mM dNTP, 10% DMSO and 2.5 U Taq DNA polymerase (Roche Applied Science, Indianapolis, Ind.). Cycle steps were as follows: denaturation (94° C.; 40 sec), annealing (55° C.; 30 sec), extension (72° C.; 60 sec), 35 cycles.

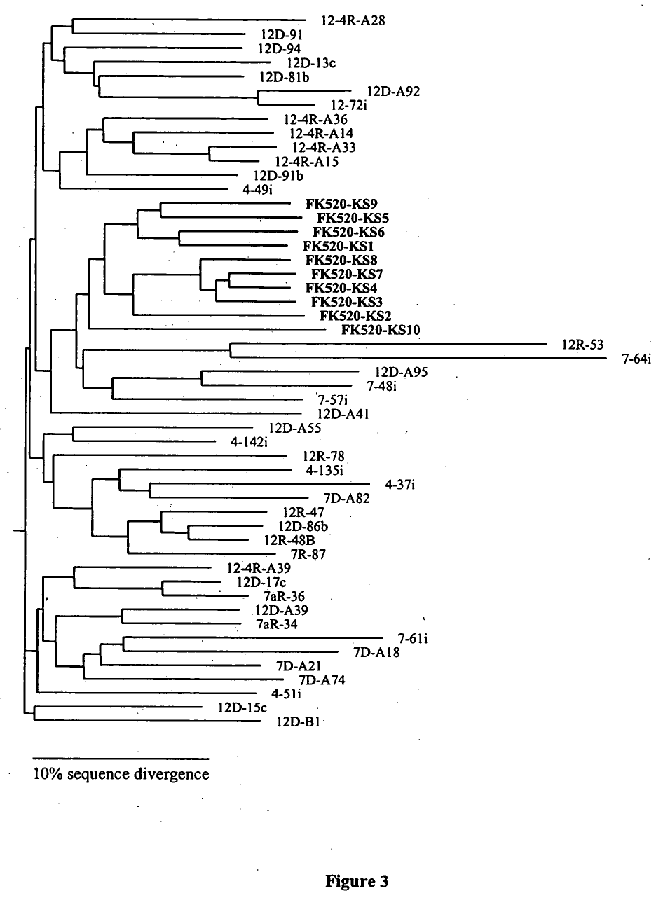

The resulting PCR reactions were electrophoresed on 1%-agarose gels. PCR products of approximately 700 bp were gel purified, polished with Pfu DNA Polymerase (Stratagene, La Jolla, Calif.) and cloned into the plasmid vector pLitmus28 (New England Biolabs, Beverley, Mass.) cut with EcoRV. 100 cloned amplimers for each strain were then sequenced using standard protocols. This procedure identified 51 and 39 unique KS amplimers from Streptomyces hygroscopicus ATCC14893 and Sorangium cellulosum Soce90, respectively. These results demonstrated that the combinations of these primers can be used to obtain a large variety of KS gene fragments from a given strain and that these primers were not biased for a small subset of PKS genes within an organism. The amplimers were compared using the program ClustalW. FIG. 3 shows a PKS similarity tree of all unique KS amplimers isolated from genomic DNA of from S. hygroscopicus 14891. Note that the FK520 KS amplimers form a distinctive cluster within the PKS tree of this strain. This indicated that the pylogentic clustering of KS sequences can correspond to distinctive PKS gene clusters in the genomes of the producer strains.

TABLE 2

Degenerate ketosynthase
(KS) primer for actinomycetes

| Primer | | Seq. |
|---|---|---|
| designation | Sequence | ID NO: |
| degKS1F | 5′-TTCGAYSCSGVSTTCTTCGSAT-3′ | 1 |
| degKS2F | 5′-GCSATGGAYCCSCARCARCGSVT-3′ | 2 |
| degKS3F | 5′-SSCTSGTSGCSMTSCAYCWSGC-3′ | 3 |
| degKS5R | 5′-GTSCCSGTSCCRTGSSCYTCSAC-3′ | 4 |
| degKS6R | 5′-TGSGYRTGSCCSAKGTTSSWCTT-3′ | 5 |
| degKS7R | 5′-ASRTGSGCRTTSGTSCCSSWSA-3′ | 6 |

TABLE 3

Degenerate ketosynthase
(KS) primer for myxobacteria.

| Primer | | Seq. |
|---|---|---|
| designation | Sequence | ID NO: |
| degKS1F.mx | 5′-TTCTTCGGSATSWSSCCSCGSGA-3′ | 7 |
| degKS3F.mx | 5′-CTSGTSKCSSTBCACCTSGCSTGC-3′ | 8 |
| degKS5R.mx | 5′-CCSAGSSWSGTSCCSGTSCCRTG-3′ | 9 |
| degKS7R.mx | 5′-TGAYRTGSGCGTTSGTSCCGSWGA-3′ | 10 |

### Example 6

### Identification of PKS Gene Fragments of Micromonospora chalcea and Streptomyces bikiniensis

Streptomyces bikiniensis and Micromonospora chalcea were subjected to degenerate PCR with the following combinations of KS primers: degKS1F+5R, degKS2F+5R and degKS3F+7R. The PCR conditions for the amplification of KS domains were as follows: A total reaction volume of 50 μl contained 100 ng of genomic DNA, 200 pmol of each primer, 0.2 mM dNTP, 10% DMSO and 2.5 U Taq DNA polymerase (Roche Applied Science, Indianapolis, Ind.). Cycle steps were as follows: denaturation (94° C.; 40 sec), annealing (55° C.; 30 sec), extension (72° C.; 60 sec), 35 cycles.

The resulting PCR reactions were electrophoresed on 1% agarose gels. PCR products of approximately 700 bp were gel purified, polished with Pfu DNA Polymerase (Stratagene, La Jolla, Calif.) and cloned into the plasmid vector pLitmus28 (New England Biolabs, Beverley, Mass.) cut with EcoRV. For each primer pair and strain, 32 amplimers were sequenced using standard protocols. This procedure gave 81 and 89 KS amplimers for Streptomyces bikiniensis and Micromonospora chalcea, respectively. Using the program ClustalW to compare the amplimers, 14 and 36 KS amplimers were found to be unique, respectively. Given that an equal number of amplimers was obtained with the same set of primers the different number of unique cloned KS amplimers in these two 16-membered macrolide producing strains implies that M. chalcea contains at least twice as many PKS genes than S. bikiniensis.

The unique KS amplimers isolated from genomic DNA of S. bikiniensis and M. chalcea were compared with the 8 KS sequences of the related tylosin PKS cluster of Streptomyces fradiae to produce phylogenetic similarity trees, using the program ClustalW. The corresponding PKS similarity trees (see FIGS. 4 and 5) identified KS amplimer Sb3/7-31 as close homolog of Tylosin KSq (23% sequence divergence),, Sb1/5-75 as close homolog of Tylosin KS3 (23% sequence divergence), Sb1/5-78 as close homolog of Tylosin KS7 (22% sequence divergence),, and Sb1/5-68, Sb1/5-75, Sb1/5-60, Sb1/5-80 and Sb1/5-67 as as close homologs of Tylosin KS1, 2, 4 and 6 (22% sequence divergence). Each of these eight KS sequences were more closely related to at least one Tylosin KS than they were to other KS sequences in the S. bikiniensis genome. Furthermore, when these KS sequences were compared to the database, they all identified Tylosin or other 16 membered macrolide PKS genes as the best BlastX hits. Therefore we concluded that these KSs correspond to the eight KSs of the chalcomycin PKS cluster. (see FIG. 5).

Analogously, Mc1/5-A55 was identified as close homolog of Tylosin KS7 (20% sequence divergence), Mc1/5-71 as close homolog of Tylosin KS5 (26% sequence divergence) and Mc2/5-A96 and Mc2/5-A67 as close homologs of Tylosin KS1, 2, 4 and 6 (25% sequence divergence) When these KS sequences were compared to the database, they all identified Tylosin or other 16 membered macrolide PKS genes as best BlastX hit. All 8 putative chalcomycin KSs could be predicted and assigned to particular KSs within the chalcomycin PKS gene cluster, whereas 4 out of 8 putative juvenimicin KSs were predicted from the phylogenetic trees (see Table 4). Note that for the purpose of obtaining specific probes or primers, only one target KS sequence per cluster needs to be identified.

TABLE 4
Similarity of Juvenimicin and chalcomycin KS sequences to the
respective tylosin KS (% identity over ca. 700 bases or translated
230 amino acid sequences).

| | Juvenimycin KSs | | | Chalcomycin KSs | | |
|---|---|---|---|---|---|---|
| | Micromonospora chalcea | | | Streptomyces bikiniensis | | |
| | KS-ID# | Protein | DNA | KS-ID# | Protein | DNA |
| KS^{Q} | not identified | — | — | Sb3/7-31 | 71 | 74 |
| KS1 | Mc2/5-A67 | 71% | 71% | Sb1/5-67 | 82 | 77 |
| KS2 | Mc2/5-A96 | 74% | 73% | Sb1/5-68 | 80 | 80 |
| KS3 | not identified | | | Sb1/5-75 | 74 | 76 |
| KS4 | not identified | — | — | Sb1/5-87 | 81 | 80 |
| KS5 | Mc1/5-A71 | 80% | 74% | Sb1/5-80 | 80 | 76 |
| KS6 | not identified | — | — | Sb1/5-60 | 76 | 77 |
| KS7 | Mc1/5-A55 | 77% | 71% | Sb1/5-78 | 74 | 74 |

### Example 7

### Cloning and Verification of Chalcomycin PKS Cosmids

Chalcomycin KSq (Sb3/7-31), KS3 (Sb1/5-75) and KS7 (Sb1/5-78) were used as probes for in-situ hybridization of a genomic cosmid library of S. bikiniensis. 15 strongly hybridizing cosmids were isolated. In order to verify chalcomycin PKS cosmids, specific primer pairs were designed for the putative chalcomycin KSq (Sb3/7-31), KS3 (Sb1/5-75) and KS7 (Sb1/5-78) (see Table 5). These primers were first tested with all 8 cloned Chalcomycin KS amplimers for their specificity. The PCR conditions for the specific amplification of these KS domains were as follows: A total reaction volume of 50 μl contained 20-100 ng of plasmid or cosmid DNA, 100 pmol of each primer, 0.2 mM dNTP, 10% DMSO and 2.5 U Taq DNA polymerase. Cycle steps were as follows: denaturation (94° C.; 40 sec), annealing (55° C. for KSq and KS3 specific primers, 65° C. for KS5 specific primers; 30 sec), extension (72° C.; 60 sec), 25 cycles.

In order to verify chalcomycin PKS cosmids, specific PCR was then performed with cosmids pkos146-185.1, pkos146-185.10 and pkos146-185.11. pkos146-185.1 gave correctly sized amplimers with KSq and KS3 but not with KS7 specific primers, whereas pkos146-185.10 gave a correctly sized amplimer with KS7 but not with KSq and KS3 specific primers. We concluded that pkos146.185.1 contained the 5′ region and pkos146.185.10 the 3′ region of the chalcomycin PKS genes. pkos146.185.11 did not give a PCR product with any of the specific primers, we concluded that this cosmid contained non-chalcomycin PKS genes. The full sequencing of cosmid pkos146.185.1 confirmed, that it comprises chalcomycin PKS from KSq to KS5 and that the KS amplimers obtained by degenerate PCR from genomic DNA were correctly assigned.

TABLE 5

Specific primer for putative
chalcomycin ketosynthases.

| Primer | | Seq |
|---|---|---|
| designation | Sequence | ID NO: |
| Sb3/7-31-F | 5′-CGTCAGCCTGATCCTCGCCGA-3′ | 11 |
| (KSq forward) | | |
| Sb3/7-31-R | 5′-TCCAGGTGGCCGACGTTCGTC-3′ | 12 |
| (KSq reverse) | | |
| Sb1/5-75-F | 5′-AACGAGATCCCGCCGGGCCTC-3′ | 13 |
| (KS3 forward) | | |
| Sb1/5-75-R | 5′-ATCACGCGTTGCTGGGCGAGG-3′ | 14 |
| (KS3 reverse) | | |
| Sb1/5-78-F | 5′-GGACGTCTGCCGGAGGGTTCC-3′ | 15 |
| (KS7 forward) | | |
| Sb1/5-78-R | 5′-GGCCCGTTGGGCACGGACAGA-3′ | 16 |
| (KS7 reverse) | | |

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

What is claimed is:

1. A method for obtaining a probe that hybridizes to a gene in a PKS gene cluster comprising:
a) identifying amplimers produced at higher frequency from amplification of cDNA from RNA of a producer cell and degenerate PCR primers that hybridize to consensus regions of gene sequences encoding a PKS domain, compared to amplification of genomic DNA of the producer cell using the same primers; and,
b) using the sequences of the amplimers selected in (a) for designing one or more probes for cloning genes in a PKS gene cluster.

2. The method of claim 1 wherein the PKS domain is selected from the group consisting of KR, AT, ACP, KR, DH, ER, and TE.

3. The method of claim 2 wherein the PKS domain is KS.

4. The method of claim 1 wherein the cDNA is prepared from RNA collected at least two different times.

5. The method of claim 1 wherein the cDNA is prepared from RNA collected from cells cultured under at least two different production conditions.

6. The method of claim 1 wherein the cDNA is prepared from RNA from cells collected prior to the time of maximum polyketide production.

7. The method of claim 1 wherein at least one probe has the sequence the same length as and identical or exactly complementary to an amplimer.

8. The method of claim 1, further comprising using the probes in screen a genomic DNA library of the producer cell for clones encoding sequence of a gene in a PKS gene cluster.

9. A method for detecting a nucleic acid encoding a PKS gene comprising hybridizing a probe obtained by the method of claim 1 to said nucleic acid and detecting the hybridization complex.

10. A method for obtaining a probe that hybridizes to a gene encoding a first PKS gene comprising:
a) determining the sequences of a plurality of amplimers prepared using degenerate PCR primers that hybridize to consensus regions of gene sequences encoding a PKS domain;
b) determining phylogenetic similarity for the amplimers in (a) and plurality of sequences encoding a domains of a gene or genes encoding one or more PKS related to said first PKS;
c) selecting the amplimer sequences from (a) that are most closely related to one or more domain-encoding sequences in (b); and,
d) using the sequences selected in (c) for designing probes that hybridize to said first PKS gene.

11. The method of claim 10 wherein the domain is selected from the group consisting of KR, AT, ACP, KR, DH, ER, and TE.

12. The method of claim 11 wherein the domain is KS.

13. The method of claim 10 wherein determining phylogenetic similarity tree is done using a computer running ClustalW software.

14. The method of claim 10 wherein the sequence of the first PKS gene is not known.

15. A method for detecting a nucleic acid encoding a PKS gene comprising hybridizing a probe obtained by the method of claim 11 to said nucleic acid and detecting the hybridization complex.
